(19) United States
(12) Patent Application Publication
(10) Pub. No.: US 2023/0309971 A1
(43) Pub. Date: Oct. 5, 2023

(54) SYSTEMS FOR CAPTURING CELLS

(71) Applicant: ARTHREX, INC., Naples, FL (US)

(72) Inventors: Robert M. HARRISON, NAPLES, FL (US); Christopher BARE, NAPLES, FL (US); Matthew WITTOCK, NAPLES, FL (US); Joshua CHARLES, NORTH FORT MEYERS, FL (US)

(73) Assignee: ARTHREX, INC., Naples, FL (US)

(21) Appl. No.: 18/043,075

(22) PCT Filed: Sep. 3, 2021
(86) PCT No.: PCT/US2021/049028

§ 371 Date: Feb. 27, 2023

Related U.S. Application Data
(60) Provisional application No. 63/074,636, filed on Sep. 4, 2020.

(51) Int. Cl. A61B 10/00; A61B 10/04; A61B 10/02

(52) U.S. Cl. CPC A61B 10/0096; A61B 10/04; A61B 10/0283

(57) ABSTRACT

Embodiments provide methods of collecting stem cells. The methods can comprise connecting the proximal opening of the in-line cage device to a second device for liberating cells from a tissue in a fluid; connecting the distal opening of the in-line cage device to a suction or aspiration apparatus; and collecting stem cells on the graft or suture material. The graft can be e.g., demineralized bone matrix (DBM), bone, soft tissue, cartilage, tendon, ligament, muscle, adipose tissue, fascia, a suture, or an artificial scaffold. The second device for liberating cells can be an arthroscopic shaver, an arthroscopic bone cutter, an arthroscopic burr, or an arthroscopic drill. The method can further comprise connecting a filter device to the in-line cage device to remove tissue fragments from the fluid.

### PRIORITY

This application claims the benefit of U.S. Ser. No. 63/074,636, filed Sep. 4, 2020, the contents of which are incorporation by reference in its entirety.

### BACKGROUND

During arthroscopic debridement and/or resection of soft tissue, cartilage, and bone, cells are liberated from their resident tissues. There is a need for devices and methods for collecting the cells, such as stem cells, that are otherwise lost as the arthroscopy fluid is evacuated.

### SUMMARY OF THE DISCLOSURE

Devices used for the resection of tissue fragments can liberate cells, such as stem cells, from their resident tissues. Arthroscopically generated cells can be used in grafts or sutures in vivo. Therefore, there is a need for improved devices and methods for collecting cells liberated during arthroscopic procedures. Such devices and methods will have substantial application in orthopedics, for example.

A cell collection device can include, for example, a container with a removable lid, an inlet port and an outlet port, a graft or suture chamber. A graft or suture material can be positioned inside the graft or suture chamber. In an embodiment a cell collection device can comprise a container with a removable lid; an inlet port and an outlet port; a graft or suture chamber comprising at least two panels, wherein a first panel comprises a first opening and a second panel comprises a second opening, wherein the graft or suture chamber is configured to divide an interior of the container into a first inner compartment and a second inner compartment; a connector that holds the at least two panels together; and a graft or suture material positioned between the first and second panels and at least partially covering the first and second openings; wherein the inlet port is configured to allow a sample to flow into the first inner compartment; and wherein the outlet port is configured to allow a sample to flow out of the second inner compartment.

The first and second openings can be at least partially aligned to allow fluid to flow from the first inner compartment through the first and second openings and into the second inner compartment. The graft material can comprise demineralized bone matrix (DBM), bone, soft tissue, cartilage, tendon, ligament, muscle, adipose tissue, fascia, a suture, or an artificial scaffold. The first opening, the second opening, or both the first and second openings can comprise a screen or filter. The container and the removable lid can be configured to screw or snap together. The graft can be attached to the first panel, the second panel, or both the first and second panels by a clip or pin. The cell collection device can further comprise a second device for liberating cells from a tissue connected to the inlet port, wherein the second device is an endoscopic shaver, an endoscopic bone cutter, an endoscopic burr, or an endoscopic drill. The cell collection device can further comprise a second device for collecting cells from a surgical site connected to the inlet port. The second device can be a suction wand. The container can comprise a clear material. The graft chamber can comprise two panels and a hinged connector, wherein the hinged connector is configured so that the two panels can open while remaining connected.

Other embodiments provide a method of collecting cells, such as stem cells, can include connecting the inlet port of any of the devices disclosed herein to a second device for liberating cells from a tissue; connecting the outlet port of the device to a suction or aspiration apparatus; and collecting cells on a graft or suture material. The graft can be demineralized bone matrix (DBM), bone, soft tissue, cartilage, tendon, ligament, muscle, adipose tissue, fascia, a suture, or an artificial scaffold. The second device for liberating cells can be an arthroscopic shaver, an arthroscopic bone cutter, an arthroscopic burr, or an arthroscopic drill. The method can further comprise connecting a filter device to the cell collection device to remove tissue fragments from the fluid. The filter device can be connected in series between the second device for liberating cells from a tissue and the cell collection device. The method can further comprise opening the removable lid to access the graft chamber and pulling the graft chamber out of the container.

Other embodiments comprise an in-line cage device comprising an elongated housing comprising inner and outer walls; a proximal opening and a distal opening; a chamber comprising at least a first screen and a second screen, wherein the first and second screens are configured such that any fluid entering the proximal opening and exiting the distal opening must pass through the first screen and the second screen; a graft or suture within the chamber; wherein the proximal opening is configured to allow a fluid to flow through the first screen into the chamber, through the second screen, and out of the distal opening. The chamber can be formed of the inner walls of the in-line cage device, the first screen, and the second screen. The graft can comprise demineralized bone matrix (DBM). The housing can comprise three or more portions that are configured to screw or snap together. The housing can comprise a first housing portion comprising a proximal end and a distal end, a second housing portion comprising proximal end and a distal end, and a third housing portion comprising a proximal end and a distal end, wherein the first housing portion can be attached and detached from the second housing portion and the second housing portion can be attached and detached from the third housing portion. The in-line cage device can further comprise a first screen at the distal end of the first housing portion or at the proximal end of the second housing portion; and a second screen at the distal end of the second housing portion or the proximal end of the third housing portion such that the first screen and second screen form a chamber when the first, second, and third housing portions are assembled into the in-line cage device. The in-line cage device can further comprise a second device for liberating cells from a tissue connected to the proximal opening, wherein the second device is an endoscopic shaver, an endoscopic bone cutter, an endoscopic burr, or an endoscopic drill. The in-line cage device can further be connected at the distal opening to a suction or aspiration apparatus.

Other embodiments provide methods of collecting stem cells. The methods can comprise connecting the proximal opening of the in-line cage device to a second device for liberating cells from a tissue in a fluid; connecting the distal opening of the in-line cage device to a suction or aspiration apparatus; and collecting stem cells on the graft or suture material. The graft can be e.g., demineralized bone matrix (DBM), bone, soft tissue, cartilage, tendon, ligament, muscle, adipose tissue, fascia, a suture, or an artificial scaffold. The second device for liberating cells can be an arthroscopic shaver, an arthroscopic bone cutter, an arthroscopic burr, or an arthroscopic drill. The method can further comprise connecting a filter device to the in-line cage device to remove tissue fragments from the fluid. The filter device can be connected in series between the second device for liberating cells from a tissue and the in-line cage device. The method can further comprise opening the chamber and pulling the graft or suture out of the housing.

### BRIEF DESCRIPTION OF THE DRAWINGS

The accompanying drawings are included to provide a further understanding of the methods and compositions of the disclosure, are incorporated in, and constitute a part of this specification. The drawings illustrate one or more embodiments of the disclosure, and together with the description serve to explain the concepts and operation of the disclosure.

FIG. 1 panels A-C show several views of a cell collection device. Panel A shows a graft or suture chamber in an opened position. Panel B shows a graft or suture chamber and in a closed position. Panel C show a cell collection device assembled with a closed graft or suture chamber.

FIG. 2 shows a view of an assembled in-line graft or suture cage device.

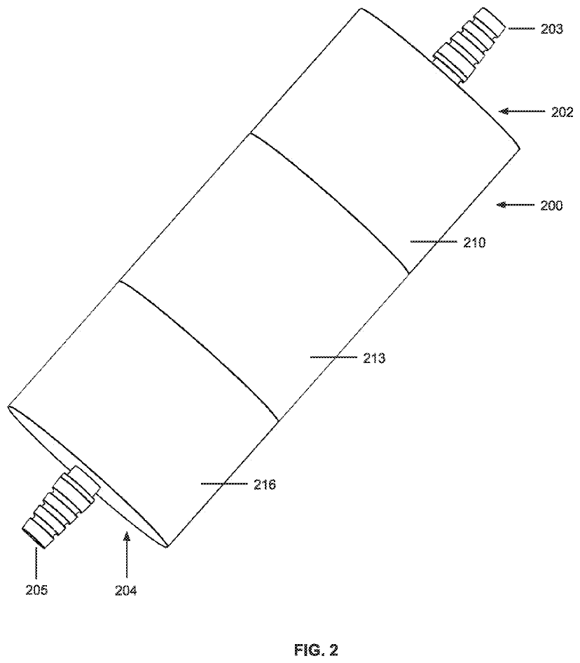

FIG. 3 shows a view of a disassembled in-line graft or suture cage device.

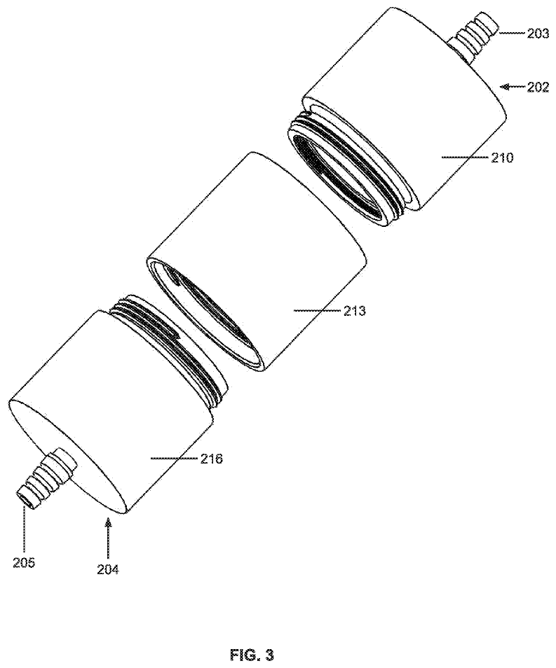

FIG. 4 shows the interior of an assembled in-line graft or suture cage device.

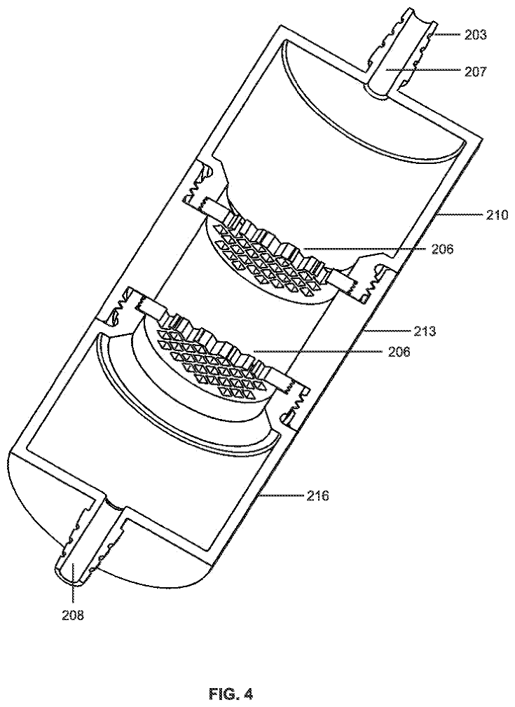

FIG. 5 shows the interior of a disassembled in-line graft or suture cage device.

FIG. 6 shows cell adherence to a collagen coated suture.

FIG. 7 shows cell adherence to an AlloSyncTM demineralized bone graft scaffold.

FIG. 8 shows cell adherence to cube and disc shaped AlloSyncTM demineralized bone scaffold.

### DETAILED DESCRIPTION

Devices for the resection of tissue fragments can liberate cells from their resident tissues, which can include stem cells. Cells, such as stem cells, collected during tissue resection procedures can be used in grafts or sutures and are therefore of interest for regenerative procedures. There is an unmet need for devices and methods for collecting certain cells, for example stem cells, among the cells liberated during arthroscopic procedures. The present devices and methods are described herein to address this unmet need.

### Cell Collection Device

A device for collecting cells from a surgical site can comprise, for example, a container 120 with a removable lid 130, an inlet port 140 and an outlet port 150, a graft or suture chamber (also referred to herein as simply “a graft chamber”) 101, comprising at least two panels, wherein a first panel 160 comprises a first opening 110 and a second panel 170 comprises a second opening 115, wherein the graft chamber is configured to divide an interior of the container into a first inner compartment 190 and a second inner compartment 195, a connector 180 that holds the at least two panels together; and a graft material positioned between the first and second panels and at least partially covering the first and second openings, wherein the inlet port is configured to allow a sample to flow into the first inner compartment, and wherein the outlet port is configured to allow a sample to flow out of the second inner compartment. FIG. 10. A graft chamber can be configured to allow a fluid to flow from a first inner compartment to a second inner compartment through a first and second openings. The container, lid, and graft chamber (other than the screen or filter) can be made up of metal, glass, polystyrene, polycarbonate, polypropylene, acrylonitrile butadiene styrene (ABS), polyethylene, or poly (methyl methacrylate) (PMMA; acrylic), or any other suitable solid, nonabsorbent material, and can be partially or fully translucent for visibility.

A first and second opening can be at least partially aligned to allow fluid to flow from a first inner compartment through first and second openings and into a second inner compartment.

A graft material can comprise a demineralized bone matrix (DBM).

A graft can be attached to a first panel, a second panel, or both a first and second panels by a clip or pin

A first opening, a second opening, or both a first and second openings can comprise a screen or filter.

A removable lid can be connected to a container by any suitable method. A container and a removable lid can be configured to screw or snap together. For example, a container can comprise an internal screw thread, and a removable lid can comprise an external screw thread. An internal screw thread of a container can be complementary to an external screw thread of a removable lid. A container can comprise an external screw thread, and a removable lid can comprise an internal screw thread. An external screw thread of the container can be complementary to an internal screw thread of a removable lid.

A second device for liberating cells from a tissue can be connected to the cell collection device, for example to an inlet port of the device. A second device can be an endoscopic shaver, an endoscopic bone cutter, an endoscopic burr, or an endoscopic drill. Another device for suction or aspiration can be connected to the device, for example to an outlet port of the device.

Methods are provided herein for collecting cells, such as stem cells. In some aspects, a cell collection device includes a container with a removable lid; an inlet port and an outlet port; a graft chamber comprising at least two panels, wherein a first panel comprises a first opening and a second panel comprises a second opening, wherein the graft chamber is configured to divide an interior of the container into a first inner compartment and a second inner compartment; a connector that holds the at least two panels together; and a graft material positioned between the first and second panels and at least partially covering the first and second openings; wherein the inlet port is configured to allow a fluid to flow into the first inner compartment and the outlet port is configured to allow a fluid to flow out of the second inner compartment. The cell collection device can be connected to a second device for liberating cells from a tissue. The outlet port of a cell collection device can be connected to a suction or aspiration apparatus. Cells, such as stem cells can be collected on a graft material.

In some aspects, a graft can be, e.g., demineralized bone matrix (DBM), bone, soft tissue, cartilage, tendon, ligament, muscle, adipose tissue, fascia, a suture, or an artificial scaffold. In other aspects, the second device for liberating cells can be an arthroscopic shaver, an arthroscopic bone cutter, an arthroscopic burr, or an arthroscopic drill.

Cells, such as stem cells, can be contained within a fluid, which can flow through openings of the graft chamber and around or through the graft material. Additionally, the method can include removing tissue fragments from the fluid prior to use of the cell collection device. The method can also include removing a removable lid to access a graft chamber and a graft material containing the cells; and pulling a graft chamber out of the container.

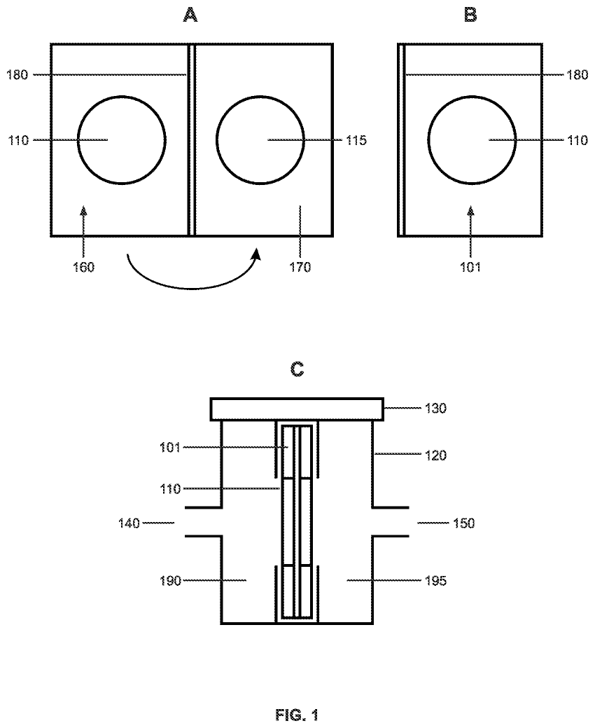

Aspects of a cell collection device are exemplified herein at inter alia, FIG. 1. Referring to FIG. 1, illustrating a device comprising a container with a removable lid, an inlet port and an outlet port, a graft chamber, and a graft material.

### Container

A container can include a generally tubular body, or any other suitable shape, with an open end. A removable lid can cover the open end. A removable lid can include a screw thread, or a press-fit, or any other suitable connector for connection to the open end. A container can include an inlet port in a wall, floor, or lid, and an outlet port at a second point in the container in a wall, floor, or lid. The inlet port and outlet ports are configured so that a graft chamber is positioned between them. An aperture running lengthwise of the container can accommodate a graft chamber, thereby defining an interior space, that can be divided by a graft chamber into a first inner compartment and a second inner compartment. An aperture can run from an open end where a removable lid can fit to an opposite end, which can be closed.

A container and a removable lid can be made of metal, glass, polystyrene, polycarbonate, polypropylene, acrylonitrile butadiene styrene (ABS), polyethylene, or poly (methyl methacrylate) (PMMA; acrylic), or any other suitable solid, nonabsorbent material, and can be partially or fully translucent for visibility.

A container and a removable lid can both include screw threads, press-fit, or other suitable connectors. For example, a container can include an internal screw thread and a removable lid can include an external screw thread, or alternatively, a container can include an external screw thread and a removable lid can include an internal screw thread. The internal screw thread and external screw thread can be designed to be complementary to one another, so that a container and a removable lid fit together to form a sealed, sterile device. Alternatively, a container and a removable lid can both include a press-fit mechanism. A press-fit mechanism can be used to fasten a removable lid to a container. A press-fit mechanism, interference fit, or friction fit is a fastening between two parts (i.e., a container and a removable lid) which can be achieved by friction after the parts are pushed together, rather than by any other means of fastening, such as a screw thread for example. The pressing operation can lead to a mechanical joint between a container and a removable lid, leading the assembly of the sterile device.

A container can comprise one or more walls (i.e., 1, 2, 3, 4, or more), depending on the shape of the body of the device. For example, a cylindrical body can comprise one wall, a triangular body can comprise three walls, a cuboid body can comprise 4 walls, etc. A container can comprise an aperture at a first terminus of the body of the container (i.e., an open end), which can host a removable lid, and a floor at the second terminus of the body of the container (i.e., a closed end opposite to the open end). Depending on the shape of the body of the container of the device, the shape of the aperture and of the floor of the container can vary. For example, a cylindrical body can comprise a round aperture and a round floor, a triangular body can comprise a triangular aperture and a triangular floor, a cuboid body can comprise a square or rectangular aperture and a square or rectangular floor, etc.

### Graft Chamber

A device can include a graft chamber that can fit within the container. A graft chamber can be configured to divide an interior of the container into a first inner compartment and a second inner compartment. An inlet port can be configured to allow a sample to flow into the first inner compartment and an outlet port can configured to allow a sample to flow out of the second inner compartment.

A graft chamber can comprise at least two panels (e.g., 2, 3, 4, 5, 6, or more). In an embodiment, a first panel can comprise a first opening and a second panel can comprise a second opening. The at least two panels can be connected with one another via one or more connectors or panels to define an interior space. A graft material can be positioned between the first and second panels within the interior space. In an embodiment, a first panel can comprise additional openings (e.g., 2, 3, 4, 5, 6 or more openings) and the second panel can comprise additional openings (e.g., 2, 3, 4, 5, 6, or more openings). The additional openings of the first panel can align with the additional openings of the second panel such that fluid can flow through the first and second openings and the additional openings. In an embodiment one or more graft materials can cover partially or fully the additional openings.

In an embodiment, a fluid can flow from a first inner compartment to a second inner compartment through the panel openings. That is, the panels of a graft chamber can block the movement of fluid from the first inner compartment to the second inner compartment other than through the panel openings.

In one embodiment, the at least two panels can be connected to one another through a connector, such as a hinge, a clip, a press fit fitting, or any other suitable connector that allows a graft chamber to be opened (FIG. 1A) and closed (FIG. 1B) to access a graft material. In another embodiment, the at least two panels can comprise a lip to fasten one or more panel to another panel. A lip can be located along one or more of the edges of one or more of the at least two panels. The presence of one or more lips on the edges of the panels can define an inner space in the graft chamber, between the two or more panels, where a graft material can be placed. In some aspects, the at least two panels of a graft chamber can be movable, and therefore can be connected to another panel through a connector, so that each panel can individually be connected or detached (i.e., a graft chamber can be completely assembled or disassembled to access a graft material).

For example, a graft chamber can comprise two panels, connected to one another through a connector so that a graft material can be sandwiched in between the two panels. For example, two panels, such as square panels or rectangular panels, can have four edges; and the two panels can be connected to one another along one of the edges (e.g., using a hinge), so that the two panels can generate a book-like structure that can be opened and closed, and wherein a graft material can fit. In another aspect, a graft chamber can comprise 6 panels. For example, two panels can each have an opening, and the additional 4 panels can have no opening. A panel with an opening, or a panel with no opening can be connected to an adjacent panel through a connector, to define a box-like structure. A graft material can be placed within the box-type structure. Two panels with an opening can be framed by 4 additional panels without openings, so that a fluid can flow inside a device through an inlet port in a first inner compartment, and through a first panel of a graft chamber having an opening, to reach the space inside graft chamber delimitated by the panels. A fluid can then flow out of a graft chamber, through a second panel having an opening to reach a second inner compartment and exit a container through an outlet port. The additional 4 panels do not have openings, so that a fluid can flow in one direction, through the graft material. In an embodiment, cells, such as stem cells, contained into the fluid are seeded onto the graft material.

A graft chamber can be of any shape, as long at the shape allows a graft chamber to fit into the container of a device. As exemplified in FIG. 1, a graft chamber can have a rectangular shape. An opening on a panel can be of any shape, and any size. For example, as exemplified in FIG. 1, in a graft chamber comprising two panels, openings can be round openings. In other aspects, an opening can be a square, a rectangle, a triangle, an oval, or a diamond. An opening in a first panel can have a same shape as an opening on a second panel of the graft chamber. For example, an opening on a first panel of a graft chamber can have a round shape and an opening on a second panel of a graft chamber can have a round shape. In other aspects, an opening on a first panel can have a different shape as compared to an opening on a second panel of the graft chamber. For example, an opening on a first panel of the graft chamber can have a round shape and an opening in a second panel of the graft chamber can have a square shape.

An opening on a first panel can have a same size as an opening on a second panel of the graft chamber. In other aspects, an opening on a first panel can have a different size as compared to the opening on a second panel of the graft chamber. In an embodiment, an opening can have an area from about 1 mm² to about 1000 mm². For example, an opening can be about 1 mm², 2 mm², 4 mm², 5 mm², 10 mm², 15 mm², 20 mm², 25 mm², 50 mm², 100 mm², 200 mm², 300 mm², 400 mm², 500 mm², 600 mm², 700 mm², 800 mm², 900 mm², 1000 mm² or more. In an embodiment an opening can be about 2, 4, 5, 10, 20, 50, 100, 200, 300, 400, 500, 600, 700, 800, 900, 1,000 mm by about 2, 4, 5, 10, 20, 50, 100, 200, 300, 400, 500, 600, 700, 800, 900, 1,000 mm. In an embodiment an opening is about 100, 200, or 500 mm by about 100, 200, or 500 mm. Where an opening is round the diameter of the opening can be about 2, 4, 5, 10, 20, 50, 100, 200, 300, 400, 500, 600, 700, 800, 900, 1,000 mm.

Any combination of shape and size of openings on a first and second panel of the graft chamber can be used, as long as the shape and size combination is suitable for cell collection on a graft material; that is, there is an alignment between the openings, such that a fluid comprising cells can flow through a first opening, through or around the graft material, and out of the graft chamber through a second opening. Openings can, for example, be aligned so that a sample can flow into a device from a first inner compartment through a graft chamber, into a second inner compartment and outside of the device.

In another embodiment, an optional panel or ‘door’ can be added to a panel having an opening. The optional panel or door can be made of the same material as the panels of the graft chamber or can be made of a different material. The optional panel or door can be used to change the size and/or the shape of an opening. The optional panel or door can, for example, be slid partially over a panel of the graft chamber.

The parts of the graft chamber (e.g., the at least two panels and the operable connector) can include polystyrene, glass, metal, polystyrene, polycarbonate, polypropylene, acrylonitrile butadiene styrene (ABS), polyethylene, or poly (methyl methacrylate) (PMMA; acrylic), or another solid, nonabsorbent material, and can be partially or fully translucent for visibility.

In an embodiment, a device can include a set of graft chambers (i.e., two or more graft chambers). For example, a device can include 2, 3, 4, or more graft chambers, so that 2, 3, 4, or more graft materials can be seeded with cells during a single procedure. A fluid comprising cells, such as stem cells, can flow from an inlet port to the first inner compartment of the device, through the two or more graft materials inserted into the two or more graft chambers, into the second inner compartment of the device and through the outlet port. In an embodiment, the two or more graft chambers can have a same opening (i.e., same size and/or same shape). In another embodiment, the two or more graft chambers can have different openings (i.e., same size and different shape, same shape and different size, or different shape and different size).

In an embodiment, an opening can include a screen or a mesh, such that a graft material can be held in place over the opening. The pore size of the screen or mesh can be adjusted depending on the size of the material being seeded. Fora DBM sponge for example, a screen or mesh can have a pore size from about 1 mm² to about 1000 mm². For example, an pore size can be about 1 mm², 2 mm², 4 mm², 5 mm², 10 mm², 15 mm², 20 mm², 25 mm², 50 mm², 100 mm², 200 mm², 300 mm², 400 mm², 500 mm², 600 mm², 700 mm², 800 mm², 900 mm², 1000 mm² or more.

In another embodiment, an interior side of a graft chamber panel can include one or more pins or clips, so that a graft material can be pinned or clipped to the interior side of a panel and does not slip away from the opening. For example, an interior side of a panel can include 1, 2, 3, 4, or more pins or clips. The one or more pins or clips can be located on one or more of the panels of the graft chamber.

### Graft Material

The graft chamber can comprise a graft material, e.g., an allograft, autograft, or synthetic graft. The graft material can be a bone material, such as a demineralized bone matrix (DBM), bone, allograft bone, soft tissue, cartilage, tendon, ligament, muscle, adipose tissue, skin or other dermal grafts, fascia, or an artificial scaffold, such as a scaffold made out of organic polysaccharides (chitin, chitosan, alginate) and/or minerals (hydroxyapatite). DBM is an allograft bone that has the inorganic mineral removed to generate an organic “collagen” matrix comprising more biologically active bone morphogenetic proteins that can modulate the differentiation of progenitor cells into osteoprogenitor cells, which are responsible for bone and cartilage formation. A graft material can be porous. The graft material can also be a synthetic mesh (such as a hernia mesh or a surgical mesh), an orthopedic polymer or composite, or suture made up of or coated with, e.g., polyglycolic acid, polylactic acid, monocyrl and polydioxanone, nylon, polyester, polvinylidenfluoride (PVDF), polypropylene, hyaluronic acid, bi-calcium phosphate, poly (D, L-lactide co-glycolide (PDGLA), poly(lactic-co-glycolic acid) (PLGA), poly(lactic acid) (PLA), poly(-L-lactic acid) (PLLA), poly (glycolic) acid (PGA), poly caprolactone (PCL), poly ethylene glycol (PEG), silk, fibroin, collagen (vitrified or recombinant), or a combination thereof.

The graft material can have any shape that is suitable to be contained inside a graft chamber. In an embodiment, the graft material can be cube, a cuboid, a cylinder, a prism, a sphere, an elongated fiber or suture, or any other shape, of any thickness commensurate with the space available in the graft chamber. For example, a cuboid graft material, having a thin thickness can be a strip, or a spheroid graft material, having a thin thickness can be a disc. For example, a graft material can be a DBM strip, or a DBM disc, or a DBM of any shape.

In an embodiment, a graft material can be a thin piece of graft material, such that the thickness of the graft material is equivalent or less than the inner space inside the graft chamber. In another embodiment, a graft material can be deformable, so that it can be compressed to fit into a graft chamber and expand back to its original state when removed from a graft chamber. That is, a graft material can have a thickness that is greater than the inner space inside the graft chamber, but that can be compressed to fit inside the graft chamber. That is, when a thick graft material is inserted in between two panels of the cell collection device, the graft material can alter the position of the two panels relatively to one another. In such a case, an additional connector can be used to hold the two or more panels connected to one another, when the graft chamber is closed. In another example, a tick graft material can be held in a graft chamber comprising more than two panels, such as 6 panels for example, in a box-like structure.

In an embodiment, a graft material size can be suitable to cover the entire surface of a graft chamber openings of a cell collection device. In other embodiments, a graft material can cover at least partially the first and second openings. That is, the graft material can cover less than the entire area of openings. For example, graft material can cover about 10%, 20%, 30%, 40%, 50%, 60%, 70%, 80%, 90% or more of the opening area.

A graft material, can fit into a graft chamber (e.g., have a thickness), or be compressed to fit into a graft chamber (e.g., have a sponge-like handling), and cover part of all of the opening area, so that a graft material can be exposed to the fluid comprising cells flowing from an inlet port into a first inner compartment, through a graft material, to a second inner compartment and outside the cell collection device through an outlet port.

To ensure that a graft material can be held over the opening, at least in part, a graft material can have a greater size than the opening, and so that independently of the position of the graft material in the graft chamber, at least a portion of the opening will be covered by the graft material. A graft material can for example have a size equivalent to an entire graft chamber. In other aspects, a graft material can have a thickness greater than the inner space defined by the panels of a graft chamber, so that when the graft chamber is closed, the graft material can be compressed, which can hold the graft material in place.

For an in-line graft or suture cage device as discussed below the graft or suture can be any size or shape that fits within the graft chamber.

### Inlet Port and Outlet Port

A cell collection device can include an inlet port and an outlet port. An inlet port can be configured so that a second device for liberating cells from a tissue at a surgical site, such as an endoscopic shaver, an endoscopic bone cutter, an endoscopic burr, an endoscopic drill, a suction wand, or the like can be connected to the device. An outlet port can be configured so that a suction or aspiration apparatus can be connected to the device. As used herein, the terms inlet port and outlet port can comprise a universal adaptor (i.e., any universal hose or fitting, regardless of its shape). The device can include an inlet port and an outlet port that are universal adapters, so that virtually any resecting or collecting device can be connected to the inlet port, and virtually any suction or aspiration device can be connected to the outlet port, regardless of the brand, connection, and characteristics of said devices. An inlet port and an outlet port are provided for the easy assembly of the device. Any suitable adaptor can be used.

An inlet port and an outlet port can be localized in any part of the device (i.e., in any part of the container or in the lid). For example, an inlet port can be localized in the removable lid, in the walls of the container, or in the floor of container. An outlet port can be localized in the removable lid, in the walls of the container, or in the floor of container.

### Assembly

An assembly can be configured to combine two or more cell collection devices (e.g., 2, 3, 4, 5 or more) as described herein or an in-line graft cage device as described below to provide for 2, 3, 4, 5, or more individual collection compartments or graft chambers. An assembly can be configured such that the two or more devices can be each connected through an inlet port or proximal opening and an outlet port or distal opening, so that the devices can be assembled in parallel. In such configuration, a fluid can enter in the two or more devices simultaneously, or individually (e.g., into a first device and then into a second device). In this embodiment a hose or other suitable tubing connects the inlet port or proximal opening of each device to a device for liberating cells from a tissue at a surgical site. In an embodiment the hose or other suitable tubing further comprises a valve. All hoses or other suitable tubing connected to each inlet port or proximal opening can be connected to the valve. The valve in turn can be connected via a hose or other suitable tubing to a device for liberating cells from a tissue at a surgical site. In an embodiment, a hose or other suitable tubing connects the outlet port or distal opening of each device to a suction or aspiration device. In an embodiment the hose or other suitable tubing connected to the outlet port or distal opening further comprises a valve. All hoses or other suitable tubing connected to each outlet port or distal opening can be connected to a valve. The valve in turn can be connected via a hose or other suitable tubing to an aspiration or suction device.

Two or more devices can be assembled in parallel, as individual compartments, and a valve can be connected so that fluid can be directed through the individual compartments at the same time or at different times. The valve can be used to bypass the fluid away from one compartment.

A valve can be used to direct fluid to and through individual devices. Based on the quantity of material required, and for ease of collection, two or more devices can be assembled in parallel. In such a configuration the fluid comprising the particulates can be diverted not only into one device but into the two or more devices, so that, e.g., larger amounts of material can be collected at once.

A valve can be used to bypass fluid away from one device and to actively direct the fluid containing the cells in a different device, or to divert said fluid from a desired device. As used herein, the valve can also be referred to as a switch. The valve or switch can be used to control the delivery of the fluid to one or more of the devices or alternate delivery back and forth in the devices.

Methods for collecting cells, such as stem cells, using an assembly of two or more cell collection or in-line graft cage devices are provided. As described above, the devices can be provided fully assembled, and ready-to-use. A method can include the connection of each the two or more devices to an adaptor, the connection of the two or more devices with a second device for liberating cells through the connection to the adaptor, and with a suction or aspiration apparatus through the connection to another adaptor.

To access the collected material, the individual devices can be separated from the assembly by disconnecting the devices from the inlet port and outlet port; the container and the removable lid can then be unscrewed or disconnected from one another, and the lid removed. The graft material can then be easily accessed, removed from the graft chamber, and processed as intended.

In another embodiment, 2 or more (e.g., 2, 3, 4, 5, or more) cell collection or in-line graft cage devices can be connected in series. That is, a hose or other suitable tubing can be used to connect the outlet port or distal opening of a first device to the inlet port or proximal opening of a second device. The outlet port or distal opening of the second device can then be connected to an aspiration or suction device. The inlet port or proximal opening of the first device can be connected to a device for liberating cells from a tissue at a surgical site.

### Methods of Use of Cell Collection Device

Methods for collecting cells, such as stem cells, are provided. A device, as described above, can be provided fully assembled. Such fully assembled device can be provided as a sterile device, ready to use in clinical or other sterile environments for cell collection. A method can include the assembly of the device with a second device for liberating cells from a tissue at a surgical site through the connection to the inlet port, and with a suction or aspiration apparatus through the connection to the outlet port.

A cell collection device can be configured such that a fluid can enter the device through an inlet port. A fluid can flow into the first inner compartment of a container through a graft chamber, into a second inner compartment of a container, and exit a device through an outlet port. Accordingly, when a cell collection device is connected to a resecting and/or collecting tissue fragments device, and to a suction or aspiration apparatus; a resecting device can liberate cells from a tissue at a surgical site including a fluid. Cells can be contained within a fluid, which can flow through the graft chamber, where they can be collected onto or in a graft material. Cells contained in a fluid can include stem cells. Stem cells are adherent cells. By directing the effluent through an opening of a graft chamber, adherent cells (i.e., the stem cells) can impact the graft material and can be captured while non-adherent cells can be evacuated through an outlet port. The stem cells liberated during resection or debridement of resident tissues can be “seeded” on a graft material such as a porous graft material. For example, bursal cells from the bursa in rotator cuff surgery or adipose tissue from the fat pad in the knee can be collected and seeded on a graft such as DBM.

A filter device can be employed proximal to a device to capture larger pieces of tissue, such that the fluid that enters into the cell collector device does not contain larger tissue pieces or fragments, but comprises cells, such as stem cells. For example, the filter device can be a tissue fragment collection device. The filter device can comprise one or more filters to remove fragments of varying size. For example, the filter device can comprise 2, 3, 4, 5 or more filters.

The filters can have a size pore ranging from about 750 μm to about 850 μm pores, so that tissue fragments having a size equal or greater than about 750 μm to about 850 μm can be removed; from about 350 μm to about 450 μm pores, so that tissue fragments having a size ranging from about 350 μm to about 850 μm can be removed; or from about 50 μm to about 100 μm pores, so that tissue fragments having a size ranging from about 50 μm to about 450 μm can be removed. In an embodiment a filter has an about 1,000, 900, 800, 700, 600, 500, 400, 300, 200, 100, 50, 40 kDa or less cut off.

To access the collected material, a removable lid and a container can be unscrewed or disconnected from one another, and a lid removed. A graft chamber can be taken out of a container directly by hand or using tweezers for example. A graft chamber can be opened, a graft material can be pulled out of a graft chamber, and processed as intended. Grafts or sutures seeded with cells using the cell collection device or the in-line graft cage device (described below) can be implanted into a mammal during any surgery, e.g. implantation and/or repair of skin, bone, nerves, tendons, cartilage, ligaments, muscle, joints, blood vessels, fat or other structures present in a mammal. The seeded cells can improve, for example, healing.

### In-Line Graft Cage Device

Another embodiment provides an in-line graft cage device. An in-line graft cage devise can comprise a cylindrical (or other suitable shaped) device that extends along a longitudinal axis between a proximal end and a distal end. The proximal end can have one or proximal openings and the distal end can have one or more distal openings. The in-line graft cage device has an inner wall or walls and an outer wall or outer wall. Provided within the device are 1, 2, 3, 4, 5, or more dividers (e.g., screens or filters) that laterally contact the inner wall of the device. That is, the outer edges of the dividers contact all points of the inner wall or walls of the device and laterally divide an interior aperture of the device into parts or portions. The one or more dividers have a multitude of openings that allow the passage of fluid and cells. For example, openings in the dividers can be any shape (e.g., square, circular, oval, etc.) and can be about 0.050 to about 0.250 inches (e.g., about 0.050, 0.100, 0.150, 0.200, 0.250 inches or any range between about 0.050 and 0.250 inches) in length, width, and/or diameter.

An in-line graft cage device can be used for seeding cells on a graft or suture and can comprise, for example, a housing including at least two dividers (e.g., filters or screens), a proximal opening with a first universal adaptor, a distal opening with a second universal adapter. The housing can be made of any material, such as, metal, glass, polystyrene, polycarbonate, polypropylene, acrylonitrile butadiene styrene (ABS), polyethylene, or poly (methyl methacrylate) (PMMA; acrylic), or any other suitable solid, nonabsorbent material, and can be partially or fully translucent for visibility. The housing can comprise 1, 2, 3, 4, 5, 6, or more portions that can be fit together. The housing portions can be assembled together to form a container. A device can be configured such that a fluid can enter the device by the proximal opening via, for example a first universal adaptor, flow from the top of the housing to the bottom of the housing through the dividers (e.g., screens or filters) and exit the device by the distal opening via, e.g., the second universal adapter.

A housing 200 can include a generally tubular body, or any other suitable shape, with an aperture running from a proximal terminus 202, where it can include a first universal adapter 203 to a distal terminus 204, where it can include a second universal adaptor 205. Two or more dividers (e.g., screens or filters) 206 (e.g., 2, 3, 4, 5, 6, 7, or more) can be present within the housing such that any fluid entering a proximal opening 207 must pass through the screens or filters 206 to exit at a distal opening 208 of the housing.

In an embodiment the two of more screens or filters form a cage or chamber 209 (also referred to herein as “a graft chamber” or “a graft and suture chamber”) within the aperture of the housing. One or more grafts or sutures (e.g., 1, 2, 3, 4, 5, 6, or more) can be placed with the cage or chamber such that liquid can pass from the proximal opening 207, through the cage or chamber 209, and out of the distal opening 208 while the graft or suture is held within the cage or chamber 209. The filter or screen 206 can also function to keep any large clumps of cells or tissue away from the graft or suture. The graft or suture can be any of the materials as described in the “Graft Materials” section above.

In some embodiments, the suture or graft can be placed into and removed from the chamber through an opening (e.g., a door or other covered opening) in a wall of the housing. In other embodiments, the housing can be split into 2, 3, 4, 5, 6, 7, 8 or more portions that can be connected together with a screw thread, or a press-fit, or other connector. Each portion of the housing can include a generally tubular body, or any other suitable shape, with an aperture running from a proximal end to a distal end, where the ends can include a screw thread, or a press-fit, or any other suitable connector.

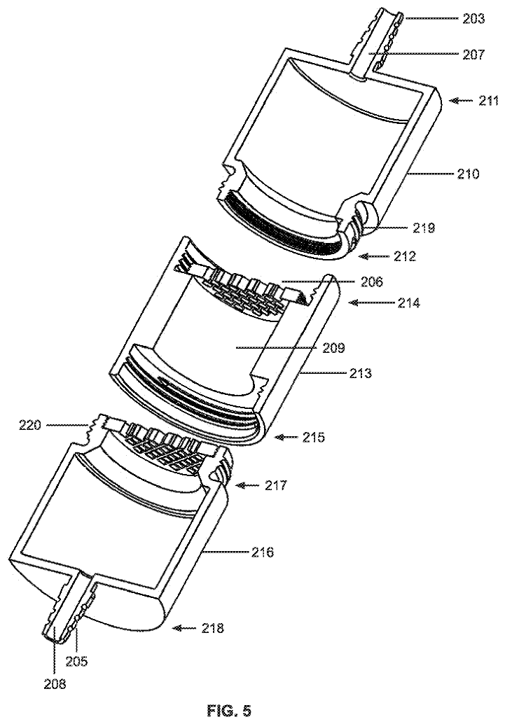

In some embodiments, a housing comprises 2, 3, 4, 5, 6, 7, 8, or more sections and 1, 2, 3, 4, 5, 6, 7, 8, or more filters or screens. For example, FIG. 5 shows a housing comprising 3 housing portions or sections and 2 filters or screens, although any suitable number of portions or filters or screens can be used. The filters or screens can be made of, for example, polycarbonate, polypropylene, acrylonitrile butadiene styrene (ABS), polyethylene, or poly (methyl methacrylate) (PMMA; acrylic). In this example, two screens form one graft or suture chamber. The housing portions can be hollow cylinders or other hollow shape (e.g., oval or square). A first housing portion 211 has a proximal end 211 and a distal end 212. The proximal end 211 of the first housing portion 211 is also the proximal end of the device when fully assembled. A second housing portion 213 has a proximal end 214 and a distal end 215. A third housing portion 216 has a proximal end 217 and a distal end 218. The distal end 218 of the third housing portion 216 is also the distal end of the device when fully assembled. The first housing portion 210 can attach to the second housing portion 213 via a screw thread 219, a press-fit, or other connector. The second housing portion 213 can attach to the third housing portion 216 via a screw thread 220, a press-fit, or other connector. A first screen or filter 206 can be present at, for example, the distal end 212 of the first housing portion 210 or the proximal end 214 of the second housing portion 213. A second screen or filter 206 can be present at, for example the distal end 215 of the second housing portion 213 or the proximal end 217 of the third housing portion 216. The first screen or filter and second screen or filter can be attached to the housing portions via a screw thread, or a press-fit, or other connector. The filters or screens can be removably attached to the housing portions. Alternatively, the filters or screens can be permanently attached to the housing portions. In the example shown in FIG. 5 a first filter or screen is removably attached to the proximal end of the second housing portion and a second filter or screen is attached to the proximal end of the third housing portion.

When the 2, 3, 4, 5, 6, 7, 8, or more housing portions are assembled into a device, the one or more filters or screens can form a chamber. One of more grafts or sutures can be placed into the chamber. Fluid entering the proximal opening can flow through one or more screens or filters into a chamber holding one or more grafts or sutures. After contacting the grafts or sutures, the fluid can run through one or more additional screens or filters and then out of a distal opening.

The device can include a proximal opening 207, at a proximal end 202 of an in-line graft cage device, and a distal opening 208 at a distal end 204 of a device. A proximal opening can be configured so that a second device for liberating cells from a tissue at a surgical site, such as an endoscopic shaver, an endoscopic bone cutter, an endoscopic burr, an endoscopic drill, a suction wand, or the like can be connected to the device. A distal opening can be configured so that a suction or aspiration apparatus can be connected to the device. A proximal opening and/or distal opening can comprise a universal adaptor (i.e., any universal hose or fitting, regardless of its shape). The device can include a proximal opening and/or distal opening that are universal adapters, so that virtually any resecting or collecting device can be connected to the proximal opening, and virtually any suction or aspiration device can be connected to the distal opening, regardless of the brand, connection, and characteristics of said devices. Any suitable adaptor can be used. A proximal opening and distal opening can be localized in any part of the device (i.e., in any part of proximal end, distal end or housing walls).

### Methods of Use of In-Line Graft Cage Device

Methods for collecting cells, such as stem cells, onto a graft or suture are provided. An in-line graft or suture cage device, as described above, can be provided fully assembled. Such fully assembled device can be provided as a sterile device, ready to use in clinical or other sterile environments. A method can include the assembly of the device with a second device for liberating cells from a tissue at a surgical site through the connection to the proximal opening, and with a suction or aspiration apparatus through the connection to the distal opening.

An in-line graft cage device can be configured such that a fluid can enter the device through a proximal opening. A fluid can flow into a housing of the device, through a graft or suture chamber, and exit the device through a distal opening. Accordingly, when an in-line graft cage device is connected to a resecting and/or collecting tissue fragments device, and to a suction or aspiration apparatus; a resecting device can liberate cells from a tissue at a surgical site including a fluid. Cells can be contained within a fluid, which can flow through the graft chamber, where they can be collected onto or in a graft or suture material. Cells contained in a fluid can include stem cells. Stem cells are adherent cells. By directing the effluent through an opening of a graft chamber, adherent cells (i.e., the stem cells) can impact the graft or suture material and can be captured while non-adherent cells can be evacuated through a distal opening. The stem cells liberated during resection or debridement of resident tissues can be “seeded” on a graft or suture material. For example, bursal cells from the bursa in rotator cuff surgery or adipose tissue from the fat pad in the knee can be collected and seeded on a graft or suture such as DBM.

A filter device can be employed proximal to an in-line graft cage device to capture larger pieces of tissue, such that the fluid that enters into the in-line graft cage device does not contain tissue pieces or fragments, but comprises cells, such as stem cells. For example, the filter device can be a tissue fragment collection device. The filter device can comprise one or more filters to remove fragments of varying size. For example, the filter device can comprise 2, 3, 4, 5 or more filters.

The filters can have a size pore ranging from about 750 μm to about 850 μm pores, so that tissue fragments having a size equal or greater than about 750 μm to about 850 μm can be removed; from about 350 μm to about 450 μm pores, so that tissue fragments having a size ranging from about 350 μm to about 850 μm can be removed; or from about 50 μm to about 100 μm pores, so that tissue fragments having a size ranging from about 50 μm to about 450 μm can be removed. In an embodiment a filter has an about 1,000, 900, 800, 700, 600, 500, 400, 300, 200, 100, 50, 40 kDa or less cut off.

To access the collected material, a housing portion can be opened, unscrewed, or disconnected such that a graft chamber is exposed. A graft chamber can be opened such that a graft or suture material can be pulled out of the graft chamber and processed as intended.

### DEFINITIONS

As used herein, a “fluid” is a liquid collected from a surgical site in a subject. The subject can be a mammal, including but not limited to primate (particularly human), equine, canine, feline, bovine, porcine, rodent, sheep, or goat. The fluid can be an isotonic solution, such as a saline solution or Ringer's lactate solution, which is commonly used to safely provide for surgical site irrigation during surgery. The fluid can include biological fluid, collected from the subject; it can be autogenic, allogenic, or xenogenic. Biological fluids include, but are not limited to, whole blood, plasma, serum, urine, saliva, mucus, cerebrospinal fluid, lymphatic fluid, seminal fluid, amniotic fluid, vitreous fluid, as well as fluid collected from cell culture of patient cells, and the like. Biological fluids also include fluids derived from tissue such as, for example, bone, bone marrow, muscle, brain, heart, liver, lung, stomach, small intestine, large intestine, colon, uterus ovary, testis, cartilage, soft tissue, skin, subcutaneous tissue, breast tissue, tissue obtained from other species, patient tissue from surgery, and the like. Biological fluids also can include, for example, bone marrow, fluids obtained from surgery, fluid filtrates, and the like.

As used herein, the term “tissue fragment” can refer to fragments, pieces, or debris obtained from a tissue during surgery. The tissue can be, e.g., soft tissue, bone or cartilage; and the tissue fragment can include bone or cartilage filtrates or fragments, bone/cartilage chips or fragments, or any other type of tissue pieces that can result from the treatment provided at the surgical site, obtained during surgery. The surgical site can be a joint, such as a knee, a shoulder, an ankle, an elbow, a hip, or a wrist. The soft tissue can be a tendon, a ligament, a muscle, adipose, or fascia.

The compositions and methods are more particularly described below and the Examples set forth herein are intended as illustrative only, as numerous modifications and variations therein will be apparent to those skilled in the art. As used in the description herein and throughout the claims that follow, the meaning of “a”, “an”, and “the” includes plural reference unless the context clearly dictates otherwise. The term “about” in association with a numerical value means that the value varies up or down by 5%. For example, for a value of about 100, means 95 to 105 (or any value between 95 and 105).

The terms used in the specification generally have their ordinary meanings in the art, within the context of the compositions and methods described herein, and in the specific context where each term is used.

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present compositions and methods have been specifically disclosed by embodiments, optional features, modification and variation of the concepts herein disclosed can be resorted to by those skilled in the art, and that such modifications and variations are considered to be within the scope of these embodiments as defined by the description and the appended claims.

Any single term, single element, single phrase, group of terms, group of phrases, or group of elements described herein can be each be specifically excluded from the claims.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods

In addition, where features or aspects of the embodiments are described in terms of Markush groups or other grouping of alternatives, those skilled in the art will recognize that the embodiments are also thereby described in terms of any individual member or subgroup of members of the Markush group or other group.

The following is provided for exemplification purposes only and is not intended to limit the scope of the embodiments described in broad terms above.

### EXAMPLES

### Example 1

An in-line cell cage tissue collector as shown in FIG. 5 was used to assess cell adhesion to various scaffolds inside the tissue collector with a continuous flow through the tissue collector.

Osteoblasts and tenocytes were cultured, harvested, and mixed in 0.9% normal saline. The cells were present at a concentration of 3,360 cells/ml. The cell suspension was circulated through the in-line cell cage tissue collector via Arthrex Dual Wave arthroscopy pump. The tissue collector contained AlloSyncTM scaffold (a demineralized bone graft) in a cube or disc shape or collagen-coated suture within the graft chamber.

The flow rate settings were:
Slow: Pump Setting at 20 ml/min for 56 min; or
Normal: Pump Setting at 30 ml/min for 5 min with 2 min lavage, for 56 min total, which resulted in 8 cycles of lavage; or
Positive control: Scaffolds inside tissue collector merely soaked in cell suspension or 56 min.

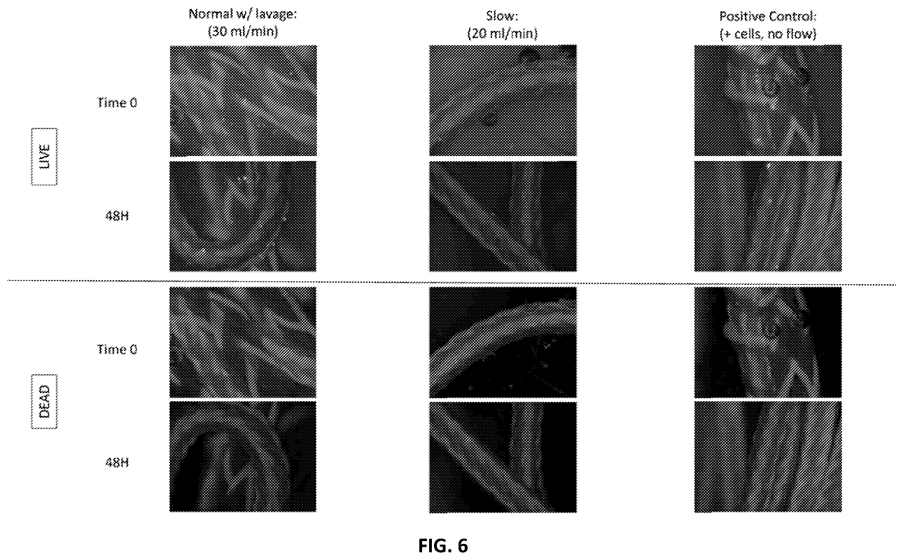

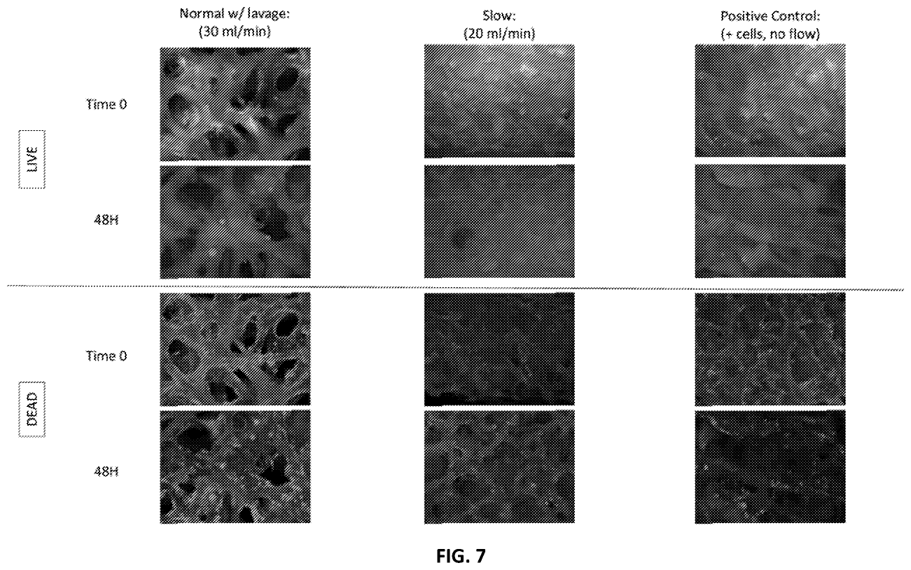

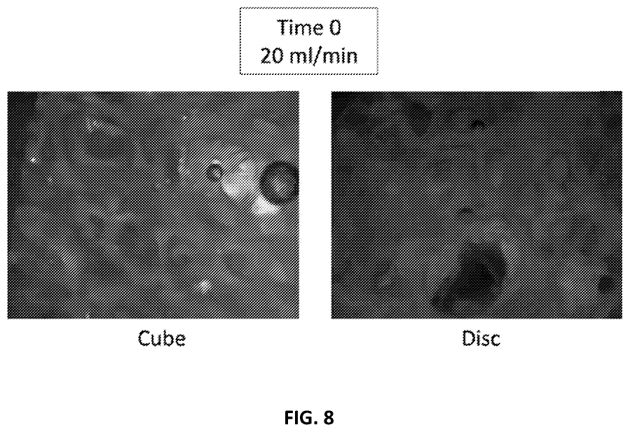

Scaffolds from each group were the stained with LIVE/DEAD (Thermo Fisher Scientific) to assess cell viability and cell adhesion to scaffold. Materials were imaged at a 4× magnification after live/dead staining and display both cell viability (green stain) and cell adhesion. FIG. 6 shows the result for the collagen coated suture. FIG. 7 shows the results for the AlloSync™ scaffold. FIG. 8 shows the results for the cube and disc shaped AlloSync™ scaffold. All sutures and scaffolds showed good live cell adherence.

At Time 0, AlloSync™ cubes showed increased cell adhesion compared to discs. This may be due to a difference in thickness between the discs and cubes. There was not a clear difference at 48 H.

Cell adherence was observed at Time 0 and 48 hours for all groups. Visually, the normal flow rate displayed higher cell adhesion than slower flow rate for all samples. Regular usage of the lavage feature, which significantly increases flow rate for a short time also increases cell adhesion. An increase of dead cells at the 48 hour time point may have been due to potential bacterial contamination.

What is claimed is:

1. A cell collection device comprising:
a container with a removable lid;
an inlet port and an outlet port;
a graft chamber comprising at least two panels, wherein a first panel comprises a first opening and a second panel comprises a second opening, wherein the graft chamber is configured to divide an interior of the container into a first inner compartment and a second inner compartment;
a connector that holds the at least two panels together; and
a graft material positioned between the first and second panels and at least partially covering the first and second openings;
wherein the inlet port is configured to allow a fluid to flow into the first inner compartment and the outlet port is configured to allow a fluid to flow out of the second inner compartment.

2. The cell collection device of claim 1, wherein the graft chamber is configured to allow a fluid to flow from the first inner compartment to the second inner compartment through the first and second openings.

3. The cell collection device of claim 2, wherein the first and second openings are at least partially aligned to allow fluid to flow from the first inner compartment through the first and second openings and into the second inner compartment.

4. The cell collection device of claim 1, wherein the graft material comprises a demineralized bone matrix (DBM), bone, cartilage, tendon, ligament, muscle, adipose tissue, fascia, a suture, or an artificial scaffold.

5. The cell collection device of claim 1, wherein the first opening, the second opening, or both the first and second openings comprise a screen or filter.

6. The cell collection device of claim 1, wherein the container and the removable lid are configured to screw or snap together.

7. The cell collection device of claim 1, wherein the graft is attached to the first panel, the second panel, or both the first and second panels by a clip or pin.

8. The cell collection device of claim 1, further comprising a second device for liberating cells from a tissue connected to the inlet port, wherein the second device is an endoscopic shaver, an endoscopic bone cutter, an endoscopic burr, or an endoscopic drill.

9. The cell collection device of claim 1, further comprising a device for aspiration or suction at the outlet port.

10. The cell collection device of claim 1, wherein the container comprises a clear material.

11. The cell collection device of claim 1, wherein the graft chamber comprises two panels and a hinged connector, wherein the hinged connector is configured so that the two panels can open while remaining connected.

12. A method of collecting stem cells comprising:
a) connecting the inlet port of the cell collection device of claim 1 to a second device for liberating cells from a tissue in a fluid;
b) connecting the outlet port of the cell collection device to a suction or aspiration apparatus; and
c) collecting stem cells on the graft material.

13. The method of claim 12, wherein the graft is demineralized bone matrix (DBM), bone, soft tissue, a suture, or an artificial scaffold.

14. The method of claim 12, wherein the second device for liberating cells is an arthroscopic shaver, an arthroscopic bone cutter, an arthroscopic burr, or an arthroscopic drill.

15. The method of claim 12, further comprising connecting a filter device to the cell collection device to remove tissue fragments from the fluid.

16. The method of claim 15, wherein the filter device is connected in series between the second device for liberating cells from a tissue and the cell collection device.

17. The method of claim 12, further comprising opening the removable lid to access the graft chamber and pulling the graft chamber out of the container.

18. The method of claim 13, wherein the soft tissue is tendon, ligament, muscle, adipose tissue, cartilage, or fascia.

19. An in-line cage device comprising:
an elongated housing comprising inner and outer walls;
a proximal opening and a distal opening;
a chamber comprising at least a first screen and a second screen, wherein the first and second screens are configured such that any fluid entering the proximal opening and exiting the distal opening must pass through the first screen and the second screen; and
a graft or suture within the chamber;
wherein the proximal opening is configured to allow a fluid to flow through the first screen into the chamber, through the second screen, and out of the distal opening.

20. The in-line cage device of claim 19, wherein the chamber is formed of the inner walls of the elongated housing, the first screen, and the second screen.

21. The in-line cage device of claim 19, wherein the graft comprises demineralized bone matrix (DBM), bone, cartilage, tendon, ligament, muscle, adipose tissue, fascia, a suture, or an artificial scaffold.

22. The in-line cage device of claim 19, wherein the elongated housing comprises three or more portions that are configured to screw or snap together.

23. The in-line cage device of claim 22, wherein the elongated housing comprises a first housing portion comprising a proximal end and a distal end, a second housing portion comprising proximal end and a distal end, and a third housing portion comprising a proximal end and a distal end, wherein the first housing portion can be attached and detached from the second housing portion and the second housing portion can be attached and detached from the third housing portion; and the in-line cage device further comprises:
the first screen at the distal end of the first housing portion or at the proximal end of the second housing portion; and the second screen at the distal end of the second housing portion or the proximal end of the third housing portion such that the first screen and second screen form a chamber when the first, second, and third housing portions are assembled into the in-line cage device.

24. The in-line cage device of claim 19, further comprising a second device for liberating cells from a tissue connected to the proximal opening, wherein the second device is an endoscopic shaver, an endoscopic bone cutter, an endoscopic burr, or an endoscopic drill.

25. The in-line cage device of claim 19, further comprising another device for aspiration or suction connected to the distal opening.

26. A method of collecting stem cells comprising:
a) connecting the proximal opening of the in-line cage device of claim 19 to a second device for liberating cells from a tissue in a fluid;
b) connecting the distal opening of the in-line cage device to a suction or aspiration apparatus; and
c) collecting stem cells on the graft or suture.

27. The method of claim 26, wherein the graft is demineralized bone matrix (DBM), bone, cartilage, tendon, ligament, muscle, adipose tissue, fascia, a suture, or an artificial scaffold.

28. The method of claim 26, wherein the second device for liberating cells is an arthroscopic shaver, an arthroscopic bone cutter, an arthroscopic burr, or an arthroscopic drill.

29. The method of claim 26, further comprising connecting a filter device to the in-line cage device to remove tissue fragments from the fluid.

30. The method of claim 29, wherein the filter device is connected in series between the second device for liberating cells from a tissue and the in-line cage device.

31. The method of claim 26, further comprising opening the chamber and pulling the graft or suture out of the chamber.
